(19) United States
(12) Patent Application Publication
(10) Pub. No.: US 2005/0074903 A1
(43) Pub. Date: Apr. 7, 2005

(54) Assay method using biochemical analysis units and assay apparatus

(75) Inventor: Yoshikazu Amano, Minami-ashigara-shi (JP)

Correspondence Address: SUGHRUE MION, PLLC, 2100 PENNSYLVANIA AVENUE, N.W., SUITE 800, WASHINGTON, DC, 20037, US

(73) Assignee: FUJI PHOTO FILM CO., LTD.

(21) Appl. No.: 10/794,026

(22) Filed: Mar. 8, 2004

(30) Foreign Application Priority Data
Oct. 3, 2003 (JP) 062710/2003

(51) Int. Cl. G01N 33/543

(52) U.S. Cl. 436/518

(57) ABSTRACT

A set of a plurality of biochemical analysis units having porous adsorptive regions, to which ligands or receptors have been bound respectively, are used simultaneously and arrayed in series with respect to a direction of flow of a reaction liquid containing at least one kind of a receptor or at least one kind of a ligand. The single same reaction liquid is forcibly caused to flow such that the reaction liquid flows across each of the porous adsorptive regions of the set of the plurality of the biochemical analysis units. The receptor or the ligand is thus subjected to specific binding with the ligands or the receptors having been bound to the porous adsorptive regions of the biochemical analysis units and is then detected by the utilization of a labeling substance.

### BACKGROUND OF THE INVENTION

1. Field of the Invention

This invention relates to an assay method and apparatus for detecting a receptor or a ligand. This invention particularly relates to an assay method and apparatus for detecting a receptor or a ligand by use of biochemical analysis units provided with porous adsorptive regions.

2. Description of the Related Art

Various micro array analysis systems and various macro array analysis systems have heretofore been used. With the micro array analysis systems and the macro array analysis systems, liquids containing ligands or receptors (i.e., the substances, which are capable of specifically binding to organism-originating substances and whose base sequences, base lengths, compositions, characteristics, and the like, are known) are spotted onto different positions on a surface of a biochemical analysis unit, such as a membrane filter, and a plurality of adsorptive regions are thereby formed on the surface of the biochemical analysis unit. Examples of the ligands or the receptors include hormones, tumor markers, enzymes, antibodies, antigens, abzymes, other proteins, nucleic acids, cDNA's, DNA's, and RNA's. Thereafter, a labeled receptor or a labeled ligand, which has been labeled with a radioactive labeling substance, a fluorescent labeling substance, a labeling substance capable of causing a chemical luminescence substrate to produce chemical luminescence when being brought into contact with the chemical luminescence substrate, or the like, is subjected to hybridization, or the like, with the ligands or the receptors, which are contained in the adsorptive regions of the biochemical analysis unit. The labeled receptor or the labeled ligand is thus specifically bound to at least one of the ligands or the receptors, which are contained in the adsorptive regions of the biochemical analysis unit. The labeled receptor or the labeled ligand is the substance, which has been sampled from an organism through extraction, isolation, or the like, or has been subjected to chemical treatment after being sampled, and which has been labeled with the radioactive labeling substance, the fluorescent labeling substance, the labeling substance capable of causing a chemical luminescence substrate to produce the chemical luminescence when being brought into contact with the chemical luminescence substrate, or the like. Examples of the labeled receptors or the labeled ligands include hormones, tumor markers, enzymes, antibodies, antigens, abzymes, other proteins, nucleic acids, DNA's, and mRNA's.

In cases where the labeled receptor or the labeled ligand has been labeled with the radioactive labeling substance, a stimulable phosphor layer of a stimulable phosphor sheet is then exposed to radiation radiated out from the radioactive labeling substance, which is contained selectively in the adsorptive regions of the biochemical analysis unit. Thereafter, the stimulable phosphor layer is exposed to stimulating rays, which cause the stimulable phosphor layer to emit light in proportion to the amount of energy stored on the stimulable phosphor layer during the exposure of the stimulable phosphor layer to the radiation. The light emitted by the stimulable phosphor layer is detected photoelectrically, and data for a biochemical analysis is thereby obtained.

In cases where the labeled receptor or the labeled ligand has been labeled with the fluorescent labeling substance, excitation light is irradiated to the adsorptive regions of the biochemical analysis unit, and the fluorescent labeling substance, which is contained selectively in the adsorptive regions of the biochemical analysis unit, is excited by the excitation light to produce fluorescence. The thus produced fluorescence is detected photoelectrically, and data for a biochemical analysis is thereby obtained.

In cases where the labeled receptor or the labeled ligand has been labeled with the labeling substance capable of causing a chemical luminescence substrate to produce the chemical luminescence when being brought into contact with the chemical luminescence substrate, the labeling substance, which is contained selectively in the adsorptive regions of the biochemical analysis unit, is brought into contact with the chemical luminescence substrate. Also, the chemical luminescence produced by the labeling substance is detected photoelectrically, and data for a biochemical analysis is thereby obtained.

The micro array analysis systems and the macro array analysis systems are described in, for example, U.S. Patent Laid-Open No. 20020061534.

With the micro array analysis systems and the macro array analysis systems described above, a large number of the adsorptive regions, to which the ligands or the receptors are bound, are capable of being formed at a high density at different positions on the surface of the biochemical analysis unit, and the labeled receptor or the labeled ligand, which has been labeled with the labeling substance, is capable of being subjected to the hybridization, or the like, with the ligands or the receptors, which have been bound to the adsorptive regions formed at a high density at different positions on the surface of the biochemical analysis unit. Therefore, the micro array analysis systems and the macro array analysis systems described above have the advantages in that a receptor or a ligand is capable of being analyzed quickly.

Heretofore, with the biochemical analysis systems using a biochemical analysis unit, the hybridization, or the like, has ordinarily been performed with a shaking technique. With the shaking technique, the biochemical analysis unit, on which the ligands or the receptors have been fixed, is put into a hybridization bag, and a reaction liquid, which contains the labeled receptor or the labeled ligand, is added into the hybridization bag. Also, vibrations are given to the hybridization bag, and the labeled receptor or the labeled ligand is thus moved through convection or diffusion within the hybridization bad. In this manner, the labeled receptor or the labeled ligand is specifically bound to at least one of the ligands or the receptors having been fixed on the biochemical analysis unit.

However, with the shaking technique described above, it is not always possible to achieve uniform contact of the hybridization reaction liquid with the plurality of the adsorptive regions, which contain the ligands or the receptors. Therefore, the problems occur in that the ligands or the receptors and the labeled receptor or the labeled ligand cannot efficiently be subjected to the hybridization. In order to solve the problems described above, the applicant proposed a technique, wherein a reaction liquid containing a labeled receptor or a labeled ligand is forcibly caused to flow across each of adsorptive regions of a biochemical analysis unit, such that the labeled receptor or the labeled ligand may penetrate sufficiently into the interior of each of the adsorptive regions of the biochemical analysis unit. The proposed technique is described in U.S. Patent Laid-Open No. 20030148543.

[Patent Literature 1] U.S. Patent Laid-Open No. 20020061534

Heretofore, one biochemical analysis unit has been used for one time of the operation for the hybridization reaction. However, the amount of the reaction liquid necessary for one time of the operation for the hybridization reaction is determined previously. Therefore, in cases where the operations for the hybridization reaction are to be performed by use of a plurality of the biochemical analysis units, it has heretofore been necessary for the amount of the reaction liquid to be increased in proportion to the number of the biochemical analysis units. Also, it has heretofore been necessary for the operation for the hybridization reaction to be iterated in accordance with the number of the biochemical analysis units used.

Accordingly, in cases where the receptor or the ligand to be analyzed is fixed to a plurality of the biochemical analysis units, or in cases where a plurality of times of experiments are to be performed in order for a mean value of measured values to be obtained, it is necessary that the amount of the reaction liquid be increased. However, in such cases, since the amount of a sample available is limited, the problems occur in that the concentration of the sample in the increased amount of the reaction liquid becomes low, and the sensitivity becomes low. Also, since the operation for the hybridization reaction is iterated a plurality of times, the problems occur in that the time required for the analysis to be performed becomes long in proportion to the number of the biochemical analysis units, and it often becomes necessary for a particular process for canceling a measurement error in each of the operations for the hybridization reaction.

### SUMMARY OF THE INVENTION

The primary object of the present invention is to provide an assay method using biochemical analysis units wherein, in cases where a receptor or a ligand to be analyzed is fixed to a plurality of biochemical analysis units, an operation for reaction is capable of being performed such that sensitivity is capable of being kept high, such that a reaction time is capable of being kept short, and such that the number of times of operations for the reaction need not be increased.

Another object of the present invention is to provide an assay apparatus for carrying out the assay method using biochemical analysis units.

The present invention provides a first assay method using biochemical analysis units, comprising the steps of:
i) obtaining a plurality of biochemical analysis units, each of the biochemical analysis units being provided with a plurality of porous adsorptive regions, to which ligands or receptors have been bound respectively,
ii) forcibly causing a reaction liquid containing at least one kind of a receptor or at least one kind of a ligand to flow such that the reaction liquid flows across each of the porous adsorptive regions of the biochemical analysis units, the receptor or the ligand being thus subjected to specific binding with the ligands or the receptors, each of which has been bound to one of the porous adsorptive regions of the biochemical analysis units, the receptor or the ligand being thereby specifically bound to at least one of the ligands, each of which has been bound to one of the porous adsorptive regions of the biochemical analysis units, or at least one of the receptors, each of which has been bound to one of the porous adsorptive regions of the biochemical analysis units, and
iii) detecting the receptor or the ligand, which has thus been specifically bound to at least one of the ligands or at least one of the receptors, by the utilization of a labeling substance,
wherein a set of a plurality of the biochemical analysis units are used simultaneously,
the set of the plurality of the biochemical analysis units are arrayed in series with respect to a direction of the flow of the reaction liquid, and
the single same reaction liquid is forcibly caused to flow such that the reaction liquid flows across each of the porous adsorptive regions of the set of the plurality of the biochemical analysis units.

The present invention also provides a second assay method using biochemical analysis units, comprising the steps of:
i) obtaining a plurality of biochemical analysis units, each of the biochemical analysis units being provided with a plurality of porous adsorptive regions, to which ligands or receptors have been bound respectively,
ii) forcibly causing a reaction liquid containing at least one kind of a labeled receptor or at least one kind of a labeled ligand, which has been labeled with a labeling substance, to flow such that the reaction liquid flows across each of the porous adsorptive regions of the biochemical analysis units, the labeled receptor or the labeled ligand being thus subjected to specific binding with the ligands or the receptors, each of which has been bound to one of the porous adsorptive regions of the biochemical analysis units, the labeled receptor or the labeled ligand being thereby specifically bound to at least one of the ligands, each of which has been bound to one of the porous adsorptive regions of the biochemical analysis units, or at least one of the receptors, each of which has been bound to one of the porous adsorptive regions of the biochemical analysis units, and
iii) detecting the labeled receptor or the labeled ligand, which has thus been specifically bound to at least one of the ligands or at least one of the receptors,
wherein a set of a plurality of the biochemical analysis units are used simultaneously,
the set of the plurality of the biochemical analysis units are arrayed in series with respect to a direction of the flow of the reaction liquid, and
the single same reaction liquid is forcibly caused to flow such that the reaction liquid flows across each of the porous adsorptive regions of the set of the plurality of the biochemical analysis units.

The present invention further provides a third assay method using biochemical analysis units, comprising the steps of:
i) obtaining a plurality of biochemical analysis units, each of the biochemical analysis units being provided with a plurality of porous adsorptive regions, to which ligands or receptors have been bound respectively,
ii) forcibly causing a reaction liquid containing at least one kind of a receptor or at least one kind of a ligand to flow such that the reaction liquid flows across each of the porous adsorptive regions of the biochemical analysis units, the receptor or the ligand being thus subjected to specific binding with the ligands or the receptors, each of which has been bound to one of the porous adsorptive regions of the biochemical analysis units, the receptor or the ligand being thereby specifically bound to at least one of the ligands, each of which has been bound to one of the porous adsorptive regions of the biochemical analysis units, or at least one of the receptors, each of which has been bound to one of the porous adsorptive regions of the biochemical analysis units,
iii) subjecting a labeled body, which has been labeled with a labeling substance, to specific binding with the receptor or the ligand having been specifically bound to at least one of the ligands, each of which has been bound to one of the porous adsorptive regions of the biochemical analysis units, or at least one of the receptors, each of which has been bound to one of the porous adsorptive regions of the biochemical analysis units, and
iv) detecting the receptor or the ligand, which has been specifically bound to at least one of the ligands or at least one of the receptors,
wherein a set of a plurality of the biochemical analysis units are used simultaneously,
the set of the plurality of the biochemical analysis units are arrayed in series with respect to a direction of the flow of the reaction liquid, and
the single same reaction liquid is forcibly caused to flow such that the reaction liquid flows across each of the porous adsorptive regions of the set of the plurality of the biochemical analysis units.

The present invention still further provides a fourth assay method using biochemical analysis units, comprising the steps of:
i) obtaining a plurality of biochemical analysis units, each of the biochemical analysis units being provided with a plurality of porous adsorptive regions, to which ligands or receptors have been bound respectively,
ii) forcibly causing a reaction liquid containing at least one kind of an auxiliary substance-bound receptor or at least one kind of an auxiliary substance-bound ligand, to which an auxiliary substance has been bound, to flow such that the reaction liquid flows across each of the porous adsorptive regions of the biochemical analysis units, the auxiliary substance-bound receptor or the auxiliary substance-bound ligand being thus subjected to specific binding with the ligands or the receptors, each of which has been bound to one of the porous adsorptive regions of the biochemical analysis units, the auxiliary substance-bound receptor or the auxiliary substance-bound ligand being thereby specifically bound to at least one of the ligands, each of which has been bound to one of the porous adsorptive regions of the biochemical analysis units, or at least one of the receptors, each of which has been bound to one of the porous adsorptive regions of the biochemical analysis units,
iii) subjecting an auxiliary substance-combinable labeling substance, which is capable of undergoing specific binding with the auxiliary substance, to specific binding with the auxiliary substance-bound receptor or the auxiliary substance-bound ligand having been specifically bound to at least one of the ligands, each of which has been bound to one of the porous adsorptive regions of the biochemical analysis units, or at least one of the receptors, each of which has been bound to one of the porous adsorptive regions of the biochemical analysis units, and
iv) detecting the auxiliary substance-bound receptor or the auxiliary substance-bound ligand, which has been specifically bound to at least one of the ligands or at least one of the receptors,
wherein a set of a plurality of the biochemical analysis units are used simultaneously,
the set of the plurality of the biochemical analysis units are arrayed in series with respect to a direction of the flow of the reaction liquid, and
the single same reaction liquid is forcibly caused to flow such that the reaction liquid flows across each of the porous adsorptive regions of the set of the plurality of the biochemical analysis units.

The first, second, third, and fourth assay methods using biochemical analysis units in accordance with the present invention should preferably be modified such that the set of the plurality of the biochemical analysis units are superposed one upon another, such that positions of the porous adsorptive regions of each of the biochemical analysis units coincide with the positions of the porous adsorptive regions of an adjacent biochemical analysis unit.

The present invention also provides an assay apparatus, comprising:
i) a reaction vessel, which is provided with a support section for releasably supporting a plurality of biochemical analysis units within the reaction vessel, each of the biochemical analysis units being provided with a plurality of porous adsorptive regions, to which ligands or receptors have been bound respectively, the reaction vessel being adapted to perform specific binding of the ligands or the receptors, each of which has been bound to one of the porous adsorptive regions of the biochemical analysis units, and
ii) flowing means for causing a reaction liquid to flow within the reaction vessel,
wherein the support section comprises a plurality of support subsections, each of which releasably supports at least one biochemical analysis unit, the plurality of the support subsections being located in series with respect to a direction of the flow of the reaction liquid.

With each of the first, second, third, and fourth assay methods using biochemical analysis units in accordance with the present invention, the set of the plurality of the biochemical analysis units are used simultaneously. The set of the plurality of the biochemical analysis units are arrayed in series with respect to the direction of the flow of the reaction liquid, and the single same reaction liquid is forcibly caused to flow such that the reaction liquid flows across each of the porous adsorptive regions of the set of the plurality of the biochemical analysis units. Therefore, in cases where the receptor or the ligand to be analyzed is fixed to a plurality of the biochemical analysis units, or in cases where a plurality of times of experiments are to be performed in order for a mean value of measured values to be obtained, it is not necessary that the amount of the reaction liquid be increased. Accordingly, the operation for the specific binding is capable of being performed with respect to the plurality of the biochemical analysis units and at a sample concentration identical with the sample concentration which is set in cases where an analysis is made with respect to one biochemical analysis unit. As a result, the sensitivity is capable of being kept high.

Also, with one time of the operation for reaction, the receptor or the ligand is capable of being subjected to the specific binding with the ligands or the receptors, each of which has been bound to one of the porous adsorptive regions of the biochemical analysis units. Therefore, the operation for the specific binding of the receptor or the ligand with the ligands or the receptors, each of which has been bound to one of the porous adsorptive regions of the biochemical analysis units, is capable of being performed within a period of time identical with the period of time which is required in cases where the operation for the specific binding is performed by use of one biochemical analysis unit.

Further, in cases where the assay operations are performed by using a plurality of the biochemical analysis units one after another, it is necessary for the analyses to be made with a measurement error in each of the assay operations being taken into consideration. However, with each of the first, second, third, and fourth assay methods using biochemical analysis units in accordance with the present invention, since the plurality of the biochemical analysis units are capable of being assayed with one time of the assay operation, the advantage is capable of being obtained in that a particular process for canceling a measurement error in each of assay operations need not be performed.

### BRIEF DESCRIPTION OF THE DRAWINGS

FIG. 1 is a schematic perspective view showing an example of a biochemical analysis unit utilized for the assay method using biochemical analysis units in accordance with the present invention,

FIG. 2 is a schematic sectional view showing an embodiment of a reactor utilized for the assay method using biochemical analysis units in accordance with the present invention, and

FIG. 3 is a schematic sectional view showing a different embodiment of a reactor utilized for the assay method using biochemical analysis units in accordance with the present invention.

### DESCRIPTION OF THE PREFERRED EMBODIMENTS

The present invention will hereinbelow be described in further detail with reference to the accompanying drawings.

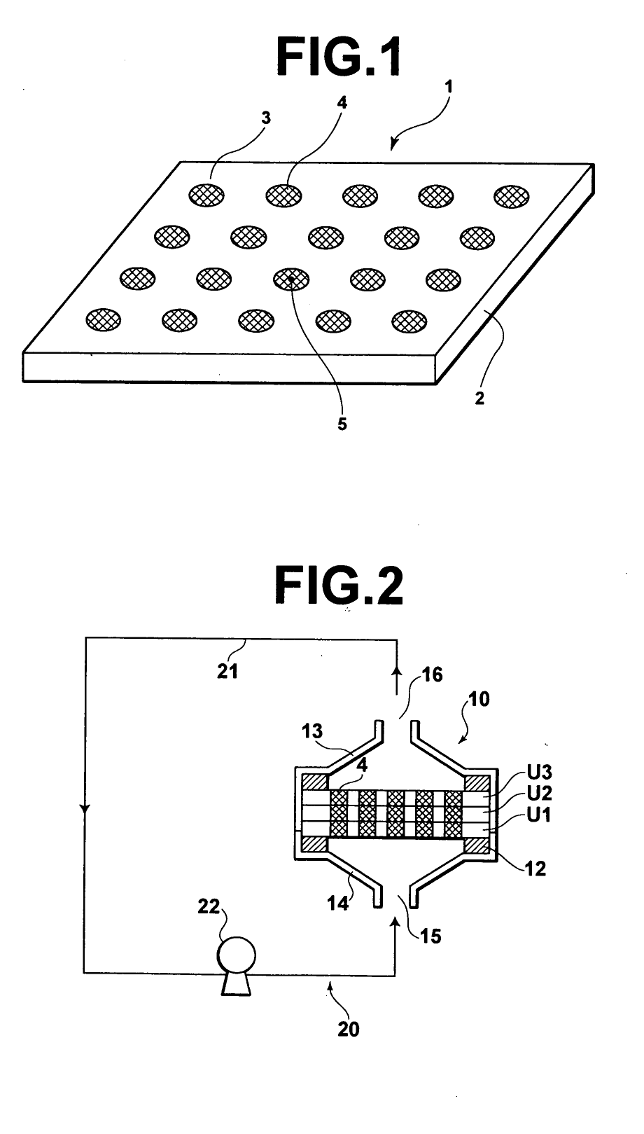

FIG. 1 is a schematic perspective view showing an example of a biochemical analysis unit utilized for the assay method using biochemical analysis units in accordance with the present invention. With reference to FIG. 1, a biochemical analysis unit 1 comprises a base plate 2, which is provided with a plurality of holes 3, 3, . . . , and a plurality of adsorptive regions 4, 4, . . . , each of which is filled in one of the holes 3, 3, . . . and comprises a porous material adhered to the base plate 2. Each of ligands or receptors, whose structures or characteristics are known, has been spotted onto one of the adsorptive regions 4, 4, . . . and has then been immobilized with treatment.

Such that light scattering may be prevented from occurring within the biochemical analysis unit 1, the base plate 2 should preferably be made from a material, which does not transmit light or which attenuates the light. The material for the formation of the base plate 2 should preferably be a metal or a ceramic material. Also, in cases where a plastic material, for which the hole making processing is capable of being performed easily, is employed as the material for the formation of the base plate 2, particles should preferably be dispersed within the plastic material, such that the light is capable of being attenuated even further.

Examples of the metals, which may be utilized preferably for the formation of the base plate 2, include copper, silver, gold, zinc, lead, aluminum, titanium, tin, chromium, iron, nickel, cobalt, tantalum, and alloys, such as stainless steel and bronze. Examples of the ceramic materials, which may be utilized preferably for the formation of the base plate 2, include alumina, zirconia, magnesia, and quartz. Examples of the plastic materials, which may be utilized preferably for the formation of the base plate 2, include polyolefins, such as a polyethylene and a polypropylene; polystyrenes; acrylic resins, such as a polymethyl methacrylate; polyvinyl chlorides; polyvinylidene chlorides; polyvinylidene fluorides; polytetrafluoroethylenes; polychlorotrifluoroethylenes; polycarbonates; polyesters, such as a polyethylene naphthalate and a polyethylene terephthalate; aliphatic polyamides, such as a 6-nylon and a 6,6-nylon; polyimides; polysulfones; polyphenylene sulfides; silicon resins, such as a polydiphenyl siloxane; phenolic resins, such as novolak; epoxy resins; polyurethanes; celluloses, such as cellulose acetate and nitrocellulose; copolymers, such as a butadiene-styrene copolymer; and blends of plastic materials.

Such that the density of the holes 3, 3, . . . made through the base plate 2 may be enhanced, the area (size) of the opening of each of the holes 3, 3, . . . may ordinarily be smaller than 5 mm². The area of the opening of each of the holes 3, 3, . . . should preferably be smaller than 1 mm², should more preferably be smaller than 0.3 mm², and should most preferably be smaller than 0.01 mm². Also, the area of the opening of each of the holes 3, 3, . . . should preferably be at least 0.001 mm².

The pitch of the holes 3, 3, . . . (i.e., the distance between the center points of two holes which are adjacent to each other) should preferably fall within the range of 0.05 mm to 3 mm. Also, the spacing between two adjacent holes 3, 3 (i.e., the shortest distance between edges of two adjacent holes 3, 3) should preferably fall within the range of 0.01 mm to 1.5 mm. The number (the array density) of the holes 3, 3, . . . may ordinarily be at least 10 holes/cm². The number (the array density) of the holes 3, 3, . . . should preferably beat least 100 holes/cm², should more preferably be at least 500 holes/cm², and should most preferably be at least 1,000 holes/cm². Also, the number (the array density) of the holes 3, 3, . . . should preferably be at most 100,000 holes/cm², and should more preferably be at most 10,000 holes/cm². The holes 3, 3, . . . need not necessarily be arrayed at equal spacing as illustrated in FIG. 1. For example, the holes 3, 3, . . . may be grouped into several number of blocks (units) comprising a plurality of holes and may be formed in units of the blocks.

In the assay method using biochemical analysis units in accordance with the present invention, as the porous material for the formation of the adsorptive regions of the biochemical analysis unit, a porous quality material or a fiber material maybe utilized preferably. The porous quality material and the fiber material may be utilized in combination in order to form the adsorptive regions of the biochemical analysis unit. In the assay method using biochemical analysis units in accordance with the present invention, the porous material, which may be utilized for the formation of the adsorptive regions of the biochemical analysis unit, may be an organic material, an inorganic material, or an organic-inorganic composite material.

The organic porous quality material, which may be utilized for the formation of the adsorptive regions of the biochemical analysis unit, may be selected from a wide variety of materials. However, the organic porous quality material should preferably be a carbon porous quality material, such as active carbon, or a porous quality material capable of forming a membrane filter. As the porous quality material capable of forming a membrane filter, a polymer soluble in a solvent should preferably be utilized. Examples of the polymers soluble in a solvent include cellulose derivatives, such as nitrocellulose, regenerated cellulose, cellulose acetate, and cellulose acetate butyrate; aliphatic polyamides, such as a 6-nylon, a 6,6-nylon, and a 4,10-nylon; polyolefins, such as a polyethylene and a polypropylene; chlorine-containing polymers, such as a polyvinyl chloride and a polyvinylidene chloride; fluorine resins, such as a polyvinylidene fluoride and a polytetrafluoride; polycarbonates; polysulfones; alginic acids and alginic acid derivatives, such as alginic acid, calcium alginate, and an alginic acid-polylysine polyion complex; and collagen. Copolymers or composite materials (mixture materials) of the above-enumerated polymers may also be utilized.

The fiber material, which may be utilized for the formation of the adsorptive regions of the biochemical analysis unit, may be selected from a wide variety of materials. Examples of the fiber materials, which may be utilized preferably, include the cellulose derivatives and the aliphatic polyamides enumerated above.

The inorganic porous quality material, which may be utilized for the formation of the adsorptive regions of the biochemical analysis unit, may be selected from a wide variety of materials. Examples of the inorganic porous quality materials, which may be utilized preferably, include metals, such as platinum, gold, iron, silver, nickel, and aluminum; oxides of metals, and the like, such as alumina, silica, titania, and zeolite; metal salts, such as hydroxyapatite and calcium sulfate; and composite materials of the above-enumerated materials.

Perforation of the plurality of the holes 3, 3, . . . through the base plate 2 may be performed with, for example, a punching technique for punching with a pin, a technique for electrical discharge machining, in which a pulsed high voltage is applied across electrodes in order to volatilize the base plate material, an etching technique, or a laser beam irradiation technique. In cases where the material of the base plate is a metal material or a plastic material, the biochemical analysis unit may be prepared with an operation for performing corona discharge or plasma discharge on the surface of the base plate, applying an adhesive agent to the surface of the base plate, and laminating the porous material for the formation of the adsorptive regions by use of means, such as a press. At the time of the lamination, the porous material for the formation of the adsorptive regions may be heated and softened, such that the adsorptive regions may be formed easily within the holes. Also, in cases where the porous material for the formation of the adsorptive regions is pressed against the base plate, the base plate and the porous material for the formation of the adsorptive regions may be divided previously into a plurality of sheets, and the plurality of the sheets may be pressed intermittently. Alternatively, a long web of the base plate and a long web of the porous material for the formation of the adsorptive regions may be conveyed continuously between two rolls.

In the assay method using biochemical analysis units in accordance with the present invention, the biochemical analysis units having been prepared by use of the material and the technique described above may be utilized. Alternatively, commercially available biochemical analysis units may be utilized. It is also possible to utilize biochemical analysis units, in which the ligands or the receptors have already been bound respectively to the porous adsorptive regions.

FIG. 2 is a schematic sectional view showing an embodiment of a reactor (a reaction apparatus), which is employed for the assay method using biochemical analysis units in accordance with the present invention. With reference to FIG. 2, the reactor comprises a reaction vessel 10 and flowing means 20. The reaction vessel 10 comprises a reaction vessel upper half 13 and a reaction vessel lower half 14. The reaction vessel upper half 13 is releasably secured to the reaction vessel lower half 14.

The reaction vessel 10 is provided with a support section for releasably supporting three biochemical analysis units U1, U2, and U3 within the reaction vessel 10, each of the biochemical analysis units U1, U2, and U3 being provided with the plurality of the porous adsorptive regions, to which the ligands or the receptors have been bound respectively. The support section comprises an upper support piece 11 and a lower support piece 12. The support section releasably supports the three biochemical analysis units U1, U2, and U3, such that the biochemical analysis units U1, U2, and U3 are superposed one upon another in close contact with one another, and such that the positions of the porous adsorptive regions of each of the biochemical analysis units U1, U2, and U3 coincide with the positions of the porous adsorptive regions of an adjacent biochemical analysis unit. When the biochemical analysis units U1, U2, and U3 are to be set within the reaction vessel 10, the reaction vessel upper half 13 is dismounted from the reaction vessel lower half 14, and the biochemical analysis units U1, U2, and U3 are set on the lower support piece 12. A bottom wall of the reaction vessel lower half 14 is provided with a liquid inlet 15, through which a reaction liquid is capable of flowing. Also, a top wall of the reaction vessel upper half 13 is provided with a liquid outlet 16, through which the reaction liquid is capable of flowing.

The flowing means 20 comprises a liquid circulating pipe 21 and a pump 22. One end of the liquid circulating pipe 21 is releasably fitted to the liquid inlet 15 of the reaction vessel 10. The other end of the liquid circulating pipe 21 is releasably fitted to the liquid outlet 16 of the reaction vessel 10. The reaction liquid is introduced by the pump 22 into the reaction vessel 10 through the liquid inlet 15. Within the reaction vessel 10, the reaction liquid is forcibly caused to flow such that the reaction liquid flows across each of the adsorptive regions 4, 4, . . . of each of the biochemical analysis units U1, U2, and U3. Thereafter, the reaction liquid is discharged through the liquid outlet 16, passes through the liquid circulating pipe 21, and circulates through the reaction vessel 10.

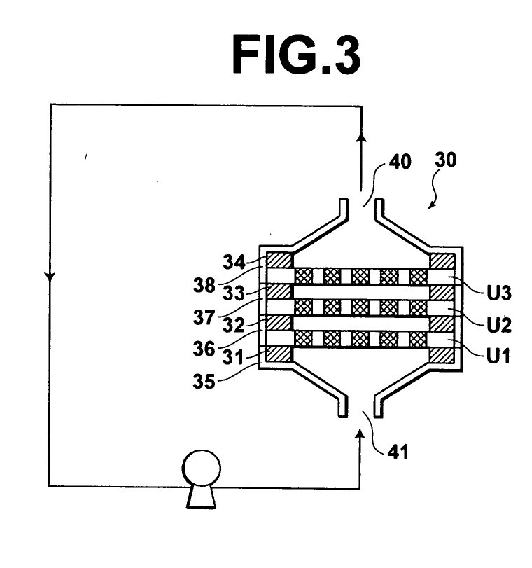

FIG. 3 is a schematic sectional view showing a different embodiment of a reactor, which is employed for the assay method using biochemical analysis units in accordance with the present invention. With reference to FIG. 3, a reaction vessel 30 is provided with a support section for releasably supporting the three biochemical analysis units U1, U2, and U3 within the reaction vessel 30, each of the biochemical analysis units U1, U2, and U3 being provided with the plurality of the porous adsorptive regions, to which the ligands or the receptors have been bound respectively. The support section comprises a first support piece 31, a second support piece 32, a third support piece 33, and a fourth support piece 34. Also, the reaction vessel 30 comprises a first vessel section 35, a second vessel section 36, a third vessel section 37, and a fourth vessel section 38. When the biochemical analysis unit U1 is to be set within the reaction vessel 30, the second vessel section 36, the third vessel section 37, and the fourth vessel section 38 are dismounted, and the biochemical analysis unit U1 is set by the first support piece 31 and the second support piece 32. When the biochemical analysis unit U2 is to be set within the reaction vessel 30, the third vessel section 37 and the fourth vessel section 38 are dismounted, and the biochemical analysis unit U1 is set by the second support piece 32 and the third support piece 33. When the biochemical analysis unit U3 is to be set within the reaction vessel 30, the fourth vessel section 38 is dismounted, and the biochemical analysis unit U3 is set by the third support piece 33 and the fourth support piece 34. A bottom wall of the first vessel section 35 is provided with a liquid inlet 41, through which a reaction liquid is capable of flowing. Also, a top wall of the fourth vessel section 38 is provided with a liquid outlet 40, through which the reaction liquid is capable of flowing. As illustrated in FIG. 3, the plurality of the biochemical analysis units need not necessarily be in close contact with one another.

In the embodiment of the reactor illustrated in FIG. 3, the plurality of the biochemical analysis units are accommodated within one reaction vessel. Alternatively, the reaction vessel may comprise a plurality of reaction subvessels, each of which accommodates one biochemical analysis unit, and the plurality of the reaction subvessels may be located in series with respect to the direction of the flow of the reaction liquid and in one flow path of the flowing reaction liquid.

In each of the embodiments of the reactor illustrated in FIG. 2 and FIG. 3, the set of the three biochemical analysis units U1, U2, and U3 are used simultaneously. However, the number of the biochemical analysis units, which are used simultaneously, is not limited to three. The number of the biochemical analysis units, which are used simultaneously, may vary in accordance with the sizes of the adsorptive regions of the biochemical analysis units and the flow rate of the reaction liquid. However, from the view point of keeping the flow of the reaction liquid, the number of the biochemical analysis units, which are used simultaneously, should preferably fall within the range of two to eight.

Also, in each of the embodiments of the reactor illustrated in FIG. 2 and FIG. 3, the pump is utilized in order to cause the reaction liquid to flow, and the reaction liquid is caused to flow and circulate in the predetermined direction. Alternatively, a reactor may be utilized, in which the reaction liquid is not circulated. For example, a reactor may be utilized in which, by the utilization of a syringe, or the like, the reaction liquid is forcibly caused to undergo reciprocal flowing across each of the adsorptive regions of the biochemical analysis units. Also, a reactor may be utilized, in which the reaction liquid merely passes through the biochemical analysis units from below (or from above).

With the assay method using biochemical analysis units in accordance with the present invention, the set of the plurality of the biochemical analysis units are used simultaneously. The set of the plurality of the biochemical analysis units are arrayed in series with respect to the direction of the flow of the reaction liquid, and the single same reaction liquid is forcibly caused to flow such that the reaction liquid flows across each of the porous adsorptive regions of the set of the plurality of the biochemical analysis units. Therefore, the ligands or the receptors, each of which has been bound to one of the porous adsorptive regions of the plurality of the biochemical analysis units, are capable of being subjected to the specific binding without the amount of the reaction liquid being increased. Accordingly, the problems do not occur in that the sample concentration is set to be low, and in that the sensitivity becomes low. Also, the operation for the specific binding of the receptor or the ligand with the ligands or the receptors, each of which has been bound to one of the porous adsorptive regions of the plurality of the biochemical analysis units, is capable of being performed within a period of time identical with the period of time which is required in cases where the operation for the specific binding is performed by use of one biochemical analysis unit.

The kinds of the ligands or the receptors, each of which is bound to one of the porous adsorptive regions of the plurality of the biochemical analysis units, may be identical among the set of the plurality of the biochemical analysis units, which are used simultaneously in the reactor. Alternatively, the kinds of the ligands or the receptors, each of which is bound to one of the porous adsorptive regions of the plurality of the biochemical analysis units, may be different among the set of the plurality of the biochemical analysis units, which are used simultaneously in the reactor. In the former cases, the number of the samples for the experiment is capable of being kept large, while the time and labor for iterating the same experiment are being eliminated. In the latter cases, the analyses of the plurality of kinds of the ligands or the receptors are capable of being made with one time of experiment, while the measurement error is being minimized.

The assay method using biochemical analysis units in accordance with the present invention is applicable broadly to various assay processes for:
i) obtaining a plurality of biochemical analysis units, each of the biochemical analysis units being provided with a plurality of porous adsorptive regions, to which ligands or receptors have been bound respectively,
ii) forcibly causing a reaction liquid containing at least one kind of a receptor or at least one kind of a ligand to flow such that the reaction liquid flows across each of the porous adsorptive regions of the biochemical analysis units, the receptor or the ligand being thus subjected to specific binding with the ligands or the receptors, each of which has been bound to one of the porous adsorptive regions of the biochemical analysis units, the receptor or the ligand being thereby specifically bound to at least one of the ligands, each of which has been bound to one of the porous adsorptive regions of the biochemical analysis units, or at least one of the receptors, each of which has been bound to one of the porous adsorptive regions of the biochemical analysis units, and
iii) detecting the receptor or the ligand, which has thus been specifically bound to at least one of the ligands or at least one of the receptors, by the utilization of a labeling substance.

In a first aspect, the assay method using biochemical analysis units in accordance with the present invention is applicable to an assay process for:
i) obtaining a plurality of biochemical analysis units, each of the biochemical analysis units being provided with a plurality of porous adsorptive regions, to which ligands or receptors have been bound respectively,
ii) forcibly causing a reaction liquid containing at least one kind of a labeled receptor or at least one kind of a labeled ligand, which has been labeled with a labeling substance, to flow such that the reaction liquid flows across each of the porous adsorptive regions of the biochemical analysis units, the labeled receptor or the labeled ligand being thus subjected to specific binding with the ligands or the receptors, each of which has been bound to one of the porous adsorptive regions of the biochemical analysis units, the labeled receptor or the labeled ligand being thereby specifically bound to at least one of the ligands, each of which has been bound to one of the porous adsorptive regions of the biochemical analysis units, or at least one of the receptors, each of which has been bound to one of the porous adsorptive regions of the biochemical analysis units, and
iii) detecting the labeled receptor or the labeled ligand, which has thus been specifically bound to at least one of the ligands or at least one of the receptors.

In such cases, the labeled receptor or the labeled ligand is the substance, which has been sampled from an organism through extraction, isolation, or the like, or has been subjected to chemical treatment after being sampled, and which has been labeled with the labeling substance. The labeled receptor or the labeled ligand is capable of undergoing the specific binding with at least one of the ligands, each of which has been bound to one of the porous adsorptive regions of the biochemical analysis unit, or at least one of the receptors, each of which has been bound to one of the porous adsorptive regions of the biochemical analysis unit. Examples of the labeled receptors or the labeled ligands include hormones, tumor markers, enzymes, antibodies, antigens, abzymes, other proteins, nucleic acids, DNA's, and mRNA's.

Examples of the labeling substances include a radioactive labeling substance, a fluorescent labeling substance, and a labeling substance capable of causing a chemical luminescence substrate to produce the chemical luminescence when being brought into contact with the chemical luminescence substrate. The labeling substance maybe a substance, which is capable of producing radiation by itself, a substance, which is capable of emitting light by itself, a substance, which is capable of forming a color by itself, or a substance, which is capable of producing fluorescence by itself when being exposed to light. Alternatively, the labeling substance may be a substance, which is capable of causing a chemical substance to emit light, to form a color, or to produce the fluorescence through, for example, decomposition or reaction of the chemical substance when being brought into contact with the chemical substance. As for the former type of the labeling substance, a radioactive isotope may be employed as the radiation producing labeling substance. Also, an acridinium ester, or the like, may be employed as the light emitting labeling substance. Further, gold colloidal particles, or the like, may be employed as the color forming labeling substance. Furthermore, fluorescein, or the like, may be employed as the fluorescent labeling substance. As the latter type of the labeling substance, an enzyme may be employed. Examples of the enzymes include alkaline phosphatase, peroxidase, luciferase, and β-galactosidase. When one of the above-enumerated enzymes acting as the labeling substance is brought into contact with a chemical luminescence substrate, a dye substrate, or a fluorescence substrate, the enzyme is capable of causing the chemical luminescence substrate to produce the chemical luminescence, causing the dye substrate to form a color, or causing the fluorescence substrate to produce the fluorescence.

By way of example, in cases where the enzyme is alkaline phosphatase, peroxidase, or luciferase, the chemical luminescence substrate may be dioxetane, luminol, or luciferin, respectively. In cases where the enzyme is alkaline phosphatase, the dye substrate may be p-nitrophenyl phosphate. In cases where the enzyme is β-galactosidase, the dye substrate may be p-nitrophenyl-β-D-galactoside, or the like. In cases where the enzyme is alkaline phosphatase, the fluorescence substrate may be 4-methylumbelliferphosphoric acid. In cases where the enzyme is peroxidase, the fluorescence substrate may be 3-(4-hydroxyphenyl)-propionic acid. In cases where the enzyme is β-galactosidase, the fluorescence substrate may be 4-methylumbellifer-β-D-galactoside, or the like.

In a second aspect, the assay method using biochemical analysis units in accordance with the present invention is applicable to an assay process for:
i) obtaining a plurality of biochemical analysis units, each of the biochemical analysis units being provided with a plurality of porous adsorptive regions, to which ligands or receptors have been bound respectively,
ii) forcibly causing a single same hybridization reaction liquid containing at least one kind of a receptor or at least one kind of a ligand to flow such that the reaction liquid flows across each of the porous adsorptive regions of the biochemical analysis units, the receptor or the ligand being thus subjected to specific binding with the ligands or the receptors, each of which has been bound to one of the porous adsorptive regions of the biochemical analysis units, the receptor or the ligand being thereby specifically bound to at least one of the ligands, each of which has been bound to one of the porous adsorptive regions of the biochemical analysis units, or at least one of the receptors, each of which has been bound to one of the porous adsorptive regions of the biochemical analysis units,
iii) subjecting a labeled body, which has been labeled with a labeling substance, to specific binding with the receptor or the ligand having been specifically bound to at least one of the ligands, each of which has been bound to one of the porous adsorptive regions of the biochemical analysis units, or at least one of the receptors, each of which has been bound to one of the porous adsorptive regions of the biochemical analysis units, and
iv) detecting the receptor or the ligand, which has been specifically bound to at least one of the ligands or at least one of the receptors.

The aforesaid second aspect of the assay method using biochemical analysis units in accordance with the present invention is the so-called sandwich technique, wherein the receptor or the ligand, which is to be detected, is sandwiched between the ligand or the receptor, which has been bound to the adsorptive region, and the labeled body. In this case, the receptor or the ligand, which is to be detected, is the substance, which has been sampled from an organism through extraction, isolation, or the like, or has been subjected to chemical treatment after being sampled, and which has been labeled with the labeling substance. The receptor or the ligand is capable of undergoing the specific binding with at least one of the ligands, each of which has been bound to one of the porous adsorptive regions of the biochemical analysis unit, or at least one of the receptors, each of which has been bound to one of the porous adsorptive regions of the biochemical analysis unit. Examples of the receptors or the ligands, which are to be detected, include hormones, tumor markers, enzymes, antibodies, antigens, abzymes, other proteins, nucleic acids, DNA's, and mRNA's.

The labeled body, which has been labeled with the labeling substance, is a body, which has been labeled with the labeling substance described above and is capable of undergoing the specific binding with a reaction site of the receptor or the ligand, which is to be detected. Examples of the labeled bodies include antigens, antibodies, hormones, tumor markers, enzymes, abzymes, other proteins, nucleic acids, cDNA's, DNA's, and RNA's, whose characteristics, compositions, structures, base sequences, base lengths, and the like, are known.

In a third aspect, the assay method using biochemical analysis units in accordance with the present invention is applicable to an assay process for:
i) obtaining a plurality of biochemical analysis units, each of the biochemical analysis units being provided with a plurality of porous adsorptive regions, to which ligands or receptors have been bound respectively,
ii) forcibly causing a reaction liquid containing at least one kind of an auxiliary substance-bound receptor or at least one kind of an auxiliary substance-bound ligand, to which an auxiliary substance has been bound, to flow such that the reaction liquid flows across each of the porous adsorptive regions of the biochemical analysis units, the auxiliary substance-bound receptor or the auxiliary substance-bound ligand being thus subjected to specific binding with the ligands or the receptors, each of which has been bound to one of the porous adsorptive regions of the biochemical analysis units, the auxiliary substance-bound receptor or the auxiliary substance-bound ligand being thereby specifically bound to at least one of the ligands, each of which has been bound to one of the porous adsorptive regions of the biochemical analysis units, or at least one of the receptors, each of which has been bound to one of the porous adsorptive regions of the biochemical analysis units,
iii) subjecting an auxiliary substance-combinable labeling substance, which is capable of undergoing specific binding with the auxiliary substance, to specific binding with the auxiliary substance-bound receptor or the auxiliary substance-bound ligand having been specifically bound to at least one of the ligands, each of which has been bound to one of the porous adsorptive regions of the biochemical analysis units, or at least one of the receptors, each of which has been bound to one of the porous adsorptive regions of the biochemical analysis units, and
iv) detecting the auxiliary substance-bound receptor or the auxiliary substance-bound ligand, which has been specifically bound to at least one of the ligands or at least one of the receptors.

The auxiliary substance is a substance capable of undergoing the binding with the auxiliary substance-combinable labeling substance. Examples of preferable auxiliary substances include antigens, such as digoxigenin, biotin, avidin, and fluorescein, and antibodies with respect to the above-enumerated antigens. Also, the auxiliary substance may be a biological binding partner, such as avidin with respect to biotin. In this case, the auxiliary substance-combinable labeling substance is a substance, which is capable of undergoing the specific binding with the auxiliary substance and has been labeled with the labeling substance described above.

The present invention will further be illustrated by the following nonlimitative example.

### EXAMPLE

### Example 1

With an etching technique, 1,600 fine holes were formed in a SUS304 sheet (acting as a base plate material sheet) having a size of 30 mm×30 mm and a thickness of 100 μm. Each of the fine holes had a circular opening region having a hole diameter of 0.3 mm. The fine holes were formed at a hole pitch of 0.45 mm and a hole spacing of 0.1 mm. The fine holes were formed with 10×10 holes being taken as one unit.

Thereafter, an adhesive agent was applied to one surface of the base plate material sheet, and the adhesive agent, which entered into the holes having been formed in the base plate material sheet, was removed by suction. The adhesive agent remaining on the surface of the base plate material sheet was then dried. Thereafter, Biodyne A (pore diameter: 0.45 μm, supplied by Paul Co., Ltd.) was superposed upon the surface of the base plate material sheet, which surface had been coated with the adhesive agent. The combination of Biodyne A and the base plate material sheet was then heated to a temperature of 150° C. and pressed under pressure such that the pressure per 1 cm² was 300 kg. Biodyne A was thus press-fitted into the fine holes of the base plate material sheet. In this manner, three biochemical analysis units 1, 2, and 3, each of which comprised a stainless steel barrier wall and the plurality of polymer-filled regions formed in the fine holes, were prepared.

Also, after a molecular weight marker pBR328/BgII, HinfI (250 nl/μg, supplied by Roche Diagnostics K.K.) having been dissolved in the TE buffer was boiled for five minutes, the liquid was cooled for one minute in an ice-bath, and the pBR328/BgII,HinfI was thus converted into a single stranded form. The thus obtained pBR328/BgII,HinfI liquid was then spotted onto the adsorptive regions of the biochemical analysis unit 1 having been prepared in the manner described above. Thereafter, with irradiation of ultraviolet light (254 nm, 33 mJ/cm²), the single stranded pBR328/BgII,HinfI was fixed to the adsorptive regions of the biochemical analysis unit 1. In the same manner as that described above, GFP-cDNA was fixed to the adsorptive regions of the biochemical analysis unit 2, and luciferase-cDNA was fixed to the adsorptive regions of the biochemical analysis unit 3.

Thereafter, 500 ng of GFP-cDNA, 100 μm digoxigenin-dUTP (alkali-stable, supplied by Roche Diagnostics K.K.), 100 μM dTTP, 500 μM dATP·dGTP·dCTP, an oligo-dT₁₂₋₁₈ primer (supplied by Invitro Gene Co.), and RNaseOUT (supplied by Invitro Gene Co.) were mixed together, and the mixture was made up to 20 μl. Also, 1 μl of a SuperScriptII reverse transcriptase (supplied by Invitro Gene Co.) was added to the mixture described above, and the resulting mixture was subjected to reaction at a temperature of 42° C. for 50 minutes. Thereafter, the reaction mixture was processed at a temperature of 70° C. for 15 minutes, and the reaction was ceased. Further, 1 μl of RNaseH (supplied by Invitro Gene Co.) was added to the reaction mixture, and the RNA was decomposed at a temperature of 37° C. for 15 minutes. The resulting mixture was then purified with ChromaSpinTE-30 (supplied by Chrontec Co.), and a digoxigenin-labeled GFP was thus obtained.

Also, 500 ng of luciferase-cDNA, 100 μM digoxigenin-dUTP (alkali-stable, supplied by Roche Diagnostics K.K.), 100 μM dTTP, 500 μM dATP·dGTP·dCTP, an oligo-dT₁₂₋₁₈ primer (supplied by Invitro Gene Co.), and RNaseOUT (supplied by Invitro Gene Co.) were mixed together, and the mixture was made up to 20 μl. Also, 1 μl of a SuperScriptII reverse transcriptase (supplied by Invitro Gene Co.) was added to the mixture described above, and the resulting mixture was subjected to reaction at a temperature of 42° C. for 50 minutes. Thereafter, the reaction mixture was processed at a temperature of 70° C. for 15 minutes, and the reaction was ceased. Further, 1 μl of RNaseH (supplied by Invitro Gene Co.) was added to the reaction mixture, and the RNA was decomposed at a temperature of 37° C. for 15 minutes. The resulting mixture was then purified with ChromaSpinTE-30 (supplied by Chrontec Co.), and a digoxigenin-labeled luciferase was thus obtained.

Thereafter, 10 pg of a digoxigenin-labeled (DIG-labeled) pBR328 (supplied by Roche Diagnostics K.K.), 10 pg of the DIG-labeled GFP having been prepared in the manner described above, and 10 pg of the DIG-labeled luciferase having been prepared in the manner described above were subjected to thermal denaturation and added to 5 ml of a hybridization buffer (6×SSC, 0.01M EDTA, 5× denhardt's solution, 0.5% SDS, 100 μg Sheared, denatured salmon sperm DNA).

The biochemical analysis units 1, 2, and 3 described above were superposed one upon another in close contact with one another, such that the positions of the holes of the base plate of each of the biochemical analysis units 1, 2, and 3 coincide with the positions of the holes of the base plate of an adjacent biochemical analysis unit. The thus obtained combination of the biochemical analysis units 1, 2, and 3 was secured to the reactor illustrated in FIG. 2, which was capable of forcibly causing a reaction liquid to flow. Also, 5 ml of a pre-hybridization buffer (the same buffer as the hybridization buffer described above) at a temperature of 65° C. was circulated within the reactor for one hour (linear speed: 0.2 cm/sec). Thereafter, the hybridization buffer, to which the DIG-labeled pBR328, DIG-labeled GFP, and the DIG-labeled luciferase had been added, was circulated within the reactor at a temperature of 65° C. for 18 hours with the technique for causing the hybridization buffer to flow across each of the adsorptive regions of each of the biochemical analysis units 1, 2, and 3. In this manner, hybridization was performed. Thereafter, a circulation washing operation was performed, wherein two washing steps were performed for five minutes per washing step by use of washing buffer 1 (2×SSC, 0.1% SDS), and wherein two washing steps were performed for five minutes per washing step by use of washing buffer 2 (0.1×SSC, 0.1% SDS). (During the circulation washing operation, the buffer temperature was 65° C.) Thereafter, a blocking buffer (DIG, described in “Wash and Block buffer Set” and supplied by Roche Diagnostics K.K.) was circulated within the reactor at room temperature for 10 minutes, and the circulation was then ceased for 50 minutes. Thereafter, an alkaline phosphatase-labeled anti-DIG antibody was diluted with the blocking buffer to a concentration of 1/10,000. The resulting dilute liquid was circulated within the reactor at room temperature for one minute, and thereafter the circulation was ceased for 60 minutes.

Thereafter, a chemiluminescent washing liquid (DIG, described in “Wash and Block buffer Set” and supplied by Roche Diagnostics K.K.) was circulated within the reactor at room temperature for 15 minutes. The operation for circulating the chemiluminescent washing liquid within the reactor was iterated three times. Thereafter, the biochemical analysis units 1, 2, and 3 were brought-into contact with a liquid containing a chemical luminescence substrate (CDP-star, ready to use, supplied by Roche Diagnostics K.K.) for one hour. Also, the chemical luminescence, which was emitted from the adsorptive regions of each of the biochemical analysis units 1, 2, and 3, was detected photoelectrically by use of a cooled CCD camera (LAS1000, supplied by Fuji Photo Film Co., Ltd.).

### Comparative Example 1

A chemical luminescence operation was performed in the same manner as that in Example 1, except that 10 pg of the DIG-labeled pBR328, 10 pg of the DIG-labeled GFP, and 10 pg of the DIG-labeled luciferase were subjected to thermal denaturation and added to 15 ml of the hybridization buffer, a 5 ml portion of the resulting hybridization buffer was used for each of the three biochemical analysis units (i.e. the biochemical analysis unit 1, to which the pBR328/BgII,HinfI had been fixed, the biochemical analysis unit 2, to which the GFP-DNA had been fixed, and the biochemical analysis unit 3, to which the luciferase-DNA had been fixed), and the three biochemical analysis units were subjected one by one to the reaction with the 5 ml portion of the hybridization buffer. Also, the chemical luminescence, which was emitted from the adsorptive regions of each of the three biochemical analysis units, was detected in the same manner as that in Example 1.

As for the biochemical analysis units used in Example 1 and Comparative Example 1, the relative values of signals listed in Table 1 below were obtained.

TABLE 1

| | Example 1 | Comp. Ex. 1 |
|---|---|---|
| Biochemical analysis unit 1 | 2.9 | 1 |
| (pBR328/BgII, HinfI) | | |
| Biochemical analysis unit 2 | 2.9 | 1 |
| (GFP-DNA) | | |
| Biochemical analysis unit 3 | 3.3 | 1 |
| (Luciferase-DNA) | | |

As clear from Table 1, in Example 1, the signals were capable of being detected with sensitivities approximately three times as high as the sensitivities obtained in Comparative Example 1.

As described above, with the assay method using biochemical analysis units in accordance with the present invention, the amount of the reaction liquid need not be increased in accordance with the number of the biochemical analysis units. Therefore, the problems do not occur in that the sample concentration is set to be low, and in that the sensitivity becomes low. In each of Example 1 and Comparative Example 1, such that the effects may be clarified, the same kind of the ligand is bound to all of the adsorptive regions of one biochemical analysis unit. Alternatively, different kinds of ligands may be bound to all of the adsorptive regions of one biochemical analysis unit. In such cases, a plurality of kinds of receptors are capable of being detected with one time of the assay operation. Also, in such cases, the kinds of the ligands or the receptors, each of which is bound to one of the porous adsorptive regions of the plurality of the biochemical analysis units, may be identical among the set of the plurality of the biochemical analysis units, which are used simultaneously in the reactor. In such cases, the number of the samples for the experiment (the “n” number) is capable of being kept large.

Further, in cases where the assay operations are performed by using a plurality of the biochemical analysis units one after another, it is necessary for the analyses to be made with a measurement error in each of the assay operations being taken into consideration. However, with the assay method using biochemical analysis units in accordance with the present invention, since the plurality of the biochemical analysis units are capable of being assayed with one time of the assay operation, the advantage is capable of being obtained in that a particular process for canceling a measurement error in each of assay operations need not be performed.

1. An assay method using biochemical analysis units, comprising the steps of:
i) obtaining a plurality of biochemical analysis units, each of the biochemical analysis units being provided with a plurality of porous adsorptive regions, to which ligands or receptors have been bound respectively,
ii) forcibly causing a reaction liquid containing at least one kind of a receptor or at least one kind of a ligand to flow such that the reaction liquid flows across each of the porous adsorptive regions of the biochemical analysis units, the receptor or the ligand being thus subjected to specific binding with the ligands or the receptors, each of which has been bound to one of the porous adsorptive regions of the biochemical analysis units, the receptor or the ligand being thereby specifically bound to at least one of the ligands, each of which has been bound to one of the porous adsorptive regions of the biochemical analysis units, or at least one of the receptors, each of which has been bound to one of the porous adsorptive regions of the biochemical analysis units, and
iii) detecting the receptor or the ligand, which has thus been specifically bound to at least one of the ligands or at least one of the receptors, by the utilization of a labeling substance,
wherein a set of a plurality of the biochemical analysis units are used simultaneously,
the set of the plurality of the biochemical analysis units are arrayed in series with respect to a direction of the flow of the reaction liquid, and
the single same reaction liquid is forcibly caused to flow such that the reaction liquid flows across each of the porous adsorptive regions of the set of the plurality of the biochemical analysis units.

2. An assay method using biochemical analysis units, comprising the steps of:
i) obtaining a plurality of biochemical analysis units, each of the biochemical analysis units being provided with a plurality of porous adsorptive regions, to which ligands or receptors have been bound respectively,
ii) forcibly causing a reaction liquid containing at least one kind of a labeled receptor or at least one kind of a labeled ligand, which has been labeled with a labeling substance, to flow such that the reaction liquid flows across each of the porous adsorptive regions of the biochemical analysis units, the labeled receptor or the labeled ligand being thus subjected to specific binding with the ligands or the receptors, each of which has been bound to one of the porous adsorptive regions of the biochemical analysis units, the labeled receptor or the labeled ligand being thereby specifically bound to at least one of the ligands, each of which has been bound to one of the porous adsorptive regions of the biochemical analysis units, or at least one of the receptors, each of which has been bound to one of the porous adsorptive regions of the biochemical analysis units, and
iii) detecting the labeled receptor or the labeled ligand, which has thus been specifically bound to at least one of the ligands or at least one of the receptors,
wherein a set of a plurality of the biochemical analysis units are used simultaneously,
the set of the plurality of the biochemical analysis units are arrayed in series with respect to a direction of the flow of the reaction liquid, and the single same reaction liquid is forcibly caused to flow such that the reaction liquid flows across each of the porous adsorptive regions of the set of the plurality of the biochemical analysis units.

3. An assay method using biochemical analysis units, comprising the steps of:
i) obtaining a plurality of biochemical analysis units, each of the biochemical analysis units being provided with a plurality of porous adsorptive regions, to which ligands or receptors have been bound respectively,
ii) forcibly causing a reaction liquid containing at least one kind of a receptor or at least one kind of a ligand to flow such that the reaction liquid flows across each of the porous adsorptive regions of the biochemical analysis units, the receptor or the ligand being thus subjected to specific binding with the ligands or the receptors, each of which has been bound to one of the porous adsorptive regions of the biochemical analysis units, the receptor or the ligand being thereby specifically bound to at least one of the ligands, each of which has been bound to one of the porous adsorptive regions of the biochemical analysis units, or at least one of the receptors, each of which has been bound to one of the porous adsorptive regions of the biochemical analysis units,
iii) subjecting a labeled body, which has been labeled with a labeling substance, to specific binding with the receptor or the ligand having been specifically bound to at least one of the ligands, each of which has been bound to one of the porous adsorptive regions of the biochemical analysis units, or at least one of the receptors, each of which has been bound to one of the porous adsorptive regions of the biochemical analysis units, and
iv) detecting the receptor or the ligand, which has been specifically bound to at least one of the ligands or at least one of the receptors,
wherein a set of a plurality of the biochemical analysis units are used simultaneously,
the set of the plurality of the biochemical analysis units are arrayed in series with respect to a direction of the flow of the reaction liquid, and
the single same reaction liquid is forcibly caused to flow such that the reaction liquid flows across each of the porous adsorptive regions of the set of the plurality of the biochemical analysis units.

4. An assay method using biochemical analysis units, comprising the steps of:
i) obtaining a plurality of biochemical analysis units, each of the biochemical analysis units being provided with a plurality of porous adsorptive regions, to which ligands or receptors have been bound respectively,
ii) forcibly causing a reaction liquid containing at least one kind of an auxiliary substance-bound receptor or at least one kind of an auxiliary substance-bound ligand, to which an auxiliary substance has been bound, to flow such that the reaction liquid flows across each of the porous adsorptive regions of the biochemical analysis units, the auxiliary substance-bound receptor or the auxiliary substance-bound ligand being thus subjected to specific binding with the ligands or the receptors, each of which has been bound to one of the porous adsorptive regions of the biochemical analysis units, the auxiliary substance-bound receptor or the auxiliary substance-bound ligand being thereby specifically bound to at least one of the ligands, each of which has been bound to one of the porous adsorptive regions of the biochemical analysis units, or at least one of the receptors, each of which has been bound to one of the porous adsorptive regions of the biochemical analysis units,
iii) subjecting an auxiliary substance-combinable labeling substance, which is capable of undergoing specific binding with the auxiliary substance, to specific binding with the auxiliary substance-bound receptor or the auxiliary substance-bound ligand having been specifically bound to at least one of the ligands, each of which has been bound to one of the porous adsorptive regions of the biochemical analysis units, or at least one of the receptors, each of which has been bound to one of the porous adsorptive regions of the biochemical analysis units, and
iv) detecting the auxiliary substance-bound receptor or the auxiliary substance-bound ligand, which has been specifically bound to at least one of the ligands or at least one of the receptors,
wherein a set of a plurality of the biochemical analysis units are used simultaneously,
the set of the plurality of the biochemical analysis units are arrayed in series with respect to a direction of the flow of the reaction liquid, and
the single same reaction liquid is forcibly caused to flow such that the reaction liquid flows across each of the porous adsorptive regions of the set of the plurality of the biochemical analysis units.

5. A method as defined in claim 1 wherein the set of the plurality of the biochemical analysis units are superposed one upon another, such that positions of the porous adsorptive regions of each of the biochemical analysis units coincide with the positions of the porous adsorptive regions of an adjacent biochemical analysis unit.

6. A method as defined in claim 2 wherein the set of the plurality of the biochemical analysis units are superposed one upon another, such that positions of the porous adsorptive regions of each of the biochemical analysis units coincide with the positions of the porous adsorptive regions of an adjacent biochemical analysis unit.

7. A method as defined in claim 3 wherein the set of the plurality of the biochemical analysis units are superposed one upon another, such that positions of the porous adsorptive regions of each of the biochemical analysis units coincide with the positions of the porous adsorptive regions of an adjacent biochemical analysis unit.

8. A method as defined in claim 4 wherein the set of the plurality of the biochemical analysis units are superposed one upon another, such that positions of the porous adsorptive regions of each of the biochemical analysis units coincide with the positions of the porous adsorptive regions of an adjacent biochemical analysis unit.

9. An assay apparatus, comprising:
i) a reaction vessel, which is provided with a support section for releasably supporting a plurality of biochemical analysis units within the reaction vessel, each of the biochemical analysis units being provided with a plurality of porous adsorptive regions, to which ligands or receptors have been bound respectively, the reaction vessel being adapted to perform specific binding of the ligands or the receptors, each of which has been bound to one of the porous adsorptive regions of the biochemical analysis units, and
ii) flowing means for causing a reaction liquid to flow within the reaction vessel,
wherein the support section comprises a plurality of support subsections, each of which releasably supports at least one biochemical analysis unit, the plurality of the support subsections being located in series with respect to a direction of the flow of the reaction liquid.

10. An apparatus as defined in claim 9 wherein the reaction vessel is adapted to perform specific binding of at least one kind of a labeled receptor or at least one kind of a labeled ligand, which has been labeled with a labeling substance, with the ligands or the receptors, each of which has been bound to one of the porous adsorptive regions of the biochemical analysis units, and
the flowing means forcibly causes a reaction liquid containing at least one kind of the labeled receptor or at least one kind of the labeled ligand to flow such that the reaction liquid flows across each of the porous adsorptive regions of the biochemical analysis units.

11. An apparatus as defined in claim 9 wherein the reaction vessel is adapted to perform:
a) specific binding of at least one kind of a receptor or at least one kind of a ligand with the ligands or the receptors, each of which has been bound to one of the porous adsorptive regions of the biochemical analysis units, and
b) specific binding of a labeled body, which has been labeled with a labeling substance, with the receptor or the ligand, which has been specifically bound to at least one of the ligands or at least one of the receptors, and
the flowing means forcibly causes a reaction liquid containing at least one kind of the receptor or at least one kind of the ligand to flow such that the reaction liquid flows across each of the porous adsorptive regions of the biochemical analysis units.

12. An apparatus as defined in claim 9 wherein the reaction vessel is adapted to perform:
a) specific binding of at least one kind of an auxiliary substance-bound receptor or at least one kind of an auxiliary substance-bound ligand, to which an auxiliary substance has been bound, with the ligands or the receptors, each of which has been bound to one of the porous adsorptive regions of the biochemical analysis units, and
b) specific binding of an auxiliary substance-combinable labeling substance, which is capable of undergoing specific binding with the auxiliary substance, with the auxiliary substance-bound receptor or the auxiliary substance-bound ligand having been specifically bound to at least one of the ligands, each of which has been bound to one of the porous adsorptive regions of the biochemical analysis units, or at least one of the receptors, each of which has been bound to one of the porous adsorptive regions of the biochemical analysis units, and
the flowing means forcibly causes a reaction liquid containing at least one kind of the auxiliary substance-bound receptor or at least one kind of the auxiliary substance-bound ligand to flow such that the reaction liquid flows across each of the porous adsorptive regions of the biochemical analysis units.

13. An apparatus as defined in claim 9 wherein the plurality of the support subsections of the support section releasably support the set of the plurality of the biochemical analysis units in a state in which the set of the plurality of the biochemical analysis units are superposed one upon another, such that positions of the porous adsorptive regions of each of the biochemical analysis units coincide with the positions of the porous adsorptive regions of an adjacent biochemical analysis unit.

14. An apparatus as defined in claim 10 wherein the plurality of the support subsections of the support section releasably support the set of the plurality of the biochemical analysis units in a state in which the set of the plurality of the biochemical analysis units are superposed one upon another, such that positions of the porous adsorptive regions of each of the biochemical analysis units coincide with the positions of the porous adsorptive regions of an adjacent biochemical analysis unit.

15. An apparatus as defined in claim 11 wherein the plurality of the support subsections of the support section releasably support the set of the plurality of the biochemical analysis units in a state in which the set of the plurality of the biochemical analysis units are superposed one upon another, such that positions of the porous adsorptive regions of each of the biochemical analysis units coincide with the positions of the porous adsorptive regions of an adjacent biochemical analysis unit.

16. An apparatus as defined in claim 12 wherein the plurality of the support subsections of the support section releasably support the set of the plurality of the biochemical analysis units in a state in which the set of the plurality of the biochemical analysis units are superposed one upon another, such that positions of the porous adsorptive regions of each of the biochemical analysis units coincide with the positions of the porous adsorptive regions of an adjacent biochemical analysis unit.
